(19) United States
(12) Patent Application Publication
(10) Pub. No.: US 2024/0157012 A1
(43) Pub. Date: May 16, 2024

(54) A SANITISING PROCESS DETECTION SYSTEM AND METHOD

(71) Applicant: INTERCEDE VENTURES LIMITED, Bristol Bristol (GB)

(72) Inventors: Janice KIELY, Chepstow Gloucestershire (GB); Richard LUXTON, Downend Bristol (GB); Graham MIMMS, Nailsea Somerset (GB)

(21) Appl. No.: 18/281,371

(22) PCT Filed: Mar. 9, 2022
(86) PCT No.: PCT/IB22/52073

§ 371 Date: Sep. 11, 2023

(30) Foreign Application Priority Data
Mar. 9, 2021 (GB) 2103261.0

(51) Int. Cl. A61L 2/28; A61L 2/18

(52) U.S. Cl. CPC A61L 2/28; A61L 2/18; A61L 2202/14

(57) ABSTRACT

This invention relates to a system and method for detecting a sanitising process and for determining the efficiency of a sanitising process.

A sanitising process detection system comprises a substrate with a sensor patch for detection of a disinfecting and/or sterilising agent.

The sensor patch produces a colour change on exposure to disinfecting and/or sterilising agent.

An optical detector is configured to detect the colour of the sensor patch at one or more predetermined time instants.

A processor compares images of the colour of the sensor patch, at a predetermined instant in time, with a datum (such as control or the colour of the sensor patch prior to exposure to the disinfecting and/or sterilising agent) and its output is used to determine the presence of a colour change and thereby indicates the efficacy of the sanitising process.

### FIELD OF THE INVENTION

This invention relates to a sanitising process detection system for detecting the efficacy of a sanitising process, for example in food, hospitality, travel and healthcare industries, aircraft, offices, warehouses and factories.

### BACKGROUND

With increased risks of contamination and cross infection there is a greater requirement for effective cleaning, hereinafter referred to as sanitising. Some sanitisation processes involve depositing antimicrobial coatings. However, despite many different sanitising and disinfectant substances being available, it is not always apparent whether a sanitising process or substance has sanitised an area, a space or a surface effectively.

The invention arose in order to overcome this problem and therefore provides a detection system for detecting the efficacy of a sanitising process.

Examples of products that are used to monitor whether levels of antimicrobials solutions are sufficient to reduce microorganisms are known.

Example of two such products are described below.

### PRIOR ART

United States patent application US 2013/0243645 (ECOLAB USA INC) discloses a colour indicator that signals when the concentration of an antimicrobial solution changes. The colour indicator can be incorporated into a variety of articles including towels, labels, containers, buckets, trays, sinks, indictor wands or strips and test kits.

Another example of a product that is used to monitor levels of antimicrobials solutions is described in United States patent application US 2013/0323854 (ECOLAB USA INC) which relates to a dye that signals when the concentration of a quaternary ammonium compound in antimicrobial solution changes.

An aim of the invention is to provide a system for detecting a sanitising process and for determining the efficiency of the sanitising process.

### SUMMARY OF INVENTION

According to a first aspect of the present invention, there is provided a sanitising process detection system comprising:
a substrate with a sensor patch which is impregnated with a chemical which produces a colour change on exposure to a disinfecting and/or sterilising agent;
an optical detector includes a processor and an optical sensor which is operative to detect the colour change; and
the processor is operative to compare the colour change with a datum to indicate efficacy of the sanitising process.

According to another aspect of the present invention, there is provided a sanitising process detection system comprising: a substrate with a material coated thereon, the material has at least one chemical which produces a colour change on exposure to a disinfecting and/or sterilising agent;
an optical detector includes a processor and an optical sensor which is operative to detect the colour change; and
the processor is operative to compare the colour change with a datum to indicate efficacy of the sanitising process.

In some systems the datum is a reference colour region on the substrate.

In some systems the substrate has a unique identifier.

In some systems the substrate has an orientation marker.

Optionally the substrate has an adhesive patch mounting portion which enables it to be stuck to items of furniture or in discreet locations, for example so as not to be visible in normal use.

The substrate may have at least one aperture configured to provide visual access to the sensor patch.

Ideally the at least one chemical is a dye which provides a colour change on exposure to quaternary ammonium compounds.

In some systems the processor includes an edge detector.

In some systems the processor includes an exposure correction means.

In some systems the processor includes a brightness correction means.

In some systems the processor includes a colour correction means.

In some systems the optical detector is included in a smartphone.

In some systems the optical sensor is included in a smartphone.

In some systems a position signal, indicative of the location of a deployed substrate, is provided. The position signal may be input or derived automatically from a source, such as a source of GPS signal data. The position signal is associated with location of the substrate and may be used to derive a score of efficacy of the sanitising process at that location.

The reference colour may be based on the colour of the sensor patch prior to exposure to the disinfecting and/or sterilising agent or to a datum or reference colour.

When provided in an electronic format an image recognition means is configured in order to sense a colour and/or hue and/or brightness and to provide a digital output.

The disinfecting and/or sterilising agent may be applied in the form of an aerosol. For example, the disinfecting and/or sterilising agent may be provided in the form of a fog or a spray to a surface to be sanitised.

The disinfecting and/or sterilising agent may include one or more of an antibacterial agent, and/or an antiviral agent, and/or an antiseptic agent, and/or an antimicrobial agent, for example: Bacoban®, Zoono® and Guardicide®.

The optical detector may be configured to detect continuously the colour of the sensor patch over a predetermined time period. Alternatively, the optical detector may be configured to detect the colour of the sensor patch at one or more time instants, after a predetermined time period of exposure to the disinfecting and/or sterilising agent.

The optical detector may be configured to provide images of the sensor patch to detect the colour of the sensor patch. The system may be configured to receive the images from the optical detector and to compare the received images to an image of the at least one sensor patch prior to exposure.

A digital memory is operable to capture images of digital frames for storage or transmission or subsequent analysis.

A database may be compiled to provide a record of deployment time and date, location and efficacy of treated (and non-treated) patches. The database therefore provides a record of efficacy of disinfecting and/or sterilising at given times and locations.

A processor is operative in order to assess and/or compare images. In a preferred embodiment a neural network is operational in accordance with software in order to provide improved, that is more accurate and more reliable, assessments of images.

The sensor patch may be composed of any suitable material. The sensor patch is preferably composed of a biodegradable material. The sensor patch may for example comprise a filter paper material, for example a medium flow rate filter paper material.

The sensor patch is preferably composed of absorbent material impregnated or coated with one or more chemicals configured to provide a colour change on exposure to disinfecting and/or sterilising agent.

The sensor patch is preferably composed of one or more of: cellulose filters and/or synthetic porous membranes.

The one or more chemicals is preferably a dye configured to provide a colour change on exposure to quaternary ammonium compounds. A non-exhaustive list of examples of such dyes that are configured to provide a colour change, includes chemicals such as bromophenol blue or Coomassie Blue®.

The sensor patch preferably further comprises one or more of: a stabilising chemical, and/or a buffer configured to maintain pH, and/or a colour reaction enhancer, including any combination thereof.

The sensor patch may be of any suitable shape, such as for example rectangular, square, oval, circular, triangular or any suitably shape to be received by the substrate or another appropriate support means.

The substrate may be configured to support and to surround at least a portion of the sensor patch thereon.

The substrate optionally defines a cavity within which the sensor patch is mounted. In some embodiments the substrate has at least one aperture configured to provide visual access to the sensor patch and/or a line of sight between the system and the sensor patch.

The substrate may further comprise one or more of: exposure correction and/or brightness correction and/or colour correction regions. The exposure/brightness correction regions may be in the form of white and black regions.

The substrate may further comprise a reference colour region. The reference colour region is preferably the colour of the sensor patch prior to use, that is, prior to exposure to the disinfecting and/or sanitizing agent.

The system may be arranged such that the system is configured to compare the colour of the patch to the colour of the reference colour region.

In one embodiment, the substrate is preferably composed of one or more biodegradable materials.

Preferably the substrate comprises a unique-identifier or indicia which is capable of identifying the substrate by way of automatic means. The unique identifier may be in the form of a bar code or QR code or number(s) or other indicia.

The optical detector may be provided by a mobile communication device, such as a smartphone which includes an imaging means and a processor.

The system may comprise a lighting condition adjustment mechanism to provide compensation to the detected colour of the sensor patch dependent on variable lighting conditions.

The system may further comprise an indication mechanism operable to provide indication as to the effectiveness of the sanitising process dependent on the colour of the sensor patch.

The system may further comprise an indication mechanism operable to provide indication as to whether the sanitising process has met one or more predefined criteria and so is deemed to be a “pass” (for example a minimum change in readout, for example representing a colour change, has been reached which indicates a predetermined minimum threshold of cleanliness/exposure to the disinfecting/sanitising agent) or a “fail” (for example when a readout has not reached a predetermined minimum threshold of cleanliness/exposure to the disinfecting/sanitising agent). The system ideally includes an algorithm to analyse the detected colour of the sensor patch to provide an indication of detection of a disinfecting and/or sanitising agent. The algorithm may be configured to provide an indication as to the effectiveness of sanitising using the disinfecting and/or sanitising agent.

The optical detector system may be provided by a smartphone with an imaging means such as a camera.

The algorithm may be configured to compensate for variable lighting conditions, dependent on the nature of the lighting.

Optionally a computer includes software which operates as a neural network to receive inputs from at least one of: red, green and blue channels; a channel indicating hue; a channel indicating saturation; and a channel indicating value.

Differences between the values from the sensor pad and printed reference colour may be derived as digital values to enable them to be stored and/or transmitted and/or proceed by appropriate electronic devices.

Training of a neural network may be performed under a range of lighting conditions encompassing different light intensities and colour temperatures.

The processor may be configured to select and apply different algorithms for different sanitising agents.

According to a third aspect of the invention, there is provided a method for assessing the efficacy of a sanitising process comprising the steps of: positioning at least one substrate which has a sensor patch which produces a colour change on exposure to a disinfecting and/or sterilising agent, the substrate has a unique identifier; associating the defined location of the at least one substrate with its unique identifier; subjecting the at least one substrate to a sanitising process for a defined time period; retrieving the at least one substrate and using an optical detector to detect a colour change of the sensor patch to derive an indication of the efficacy of the sanitising process at the location at which the substrate was positioned.

According to a further aspect of the present invention, there is provided a substrate for use with a sanitising process detection system, the substrate has a sensor patch supported thereon, the sensor patch comprises an absorbent material impregnated with at least one chemical which produces a colour change on exposure to a disinfecting and/or sterilising agent, characterised in that the substrate has an identifier.

According to a yet further aspect of the present invention, there is provided a substrate for use with a sanitising process detection system, the substrate a material coated thereon with at least one chemical which produces a colour change on exposure to a disinfecting and/or sterilising agent, characterised in that the substrate has an identifier.

The method may further comprise introducing a stabilising material for stabilising the dye on the material.

Embodiments of the present invention, will now be described in more detail, and with reference to the following Figures, in which:

### BRIEF DESCRIPTION OF FIGURES

FIG. 1 is a schematic illustration of a first embodiment of the sanitising process detection system of the present invention;

FIG. 2 is a schematic illustration of a second embodiment of the sanitising process detection system of the present invention;

FIGS. 3 and 4 show key steps in the method of verifying the sanitisation process;

FIG. 5 is a flow diagram of a preferred embodiment of the method showing steps of image alignment, colour testing using a neural network to assess the sanitisation process;

FIG. 6 is an example of a substrate with a test patch, control patch unique identifier and an orientation aid;

FIG. 7 shows an example of a template matching data for a template over an image;

FIG. 8 shows an example of template matching data of pixels in an image with good matches with the template;

FIGS. 9 and 10 show two outlines of an images of test panels: FIG. 9 is smaller than a search area and FIG. 10 is larger;

FIG. 11 shows lines obtained using alignment software to orient image date derived from a substrate;

FIGS. 12A and 12B show examples of images of two control panels located on the substrate which are used to colour correct image data;

FIG. 13 shows an example of an image of an array of substrates used as training sets; and

FIG. 14 is a diagrammatical representation of one embodiment of the system.

### DETAILED DESCRIPTION OF FIGURES

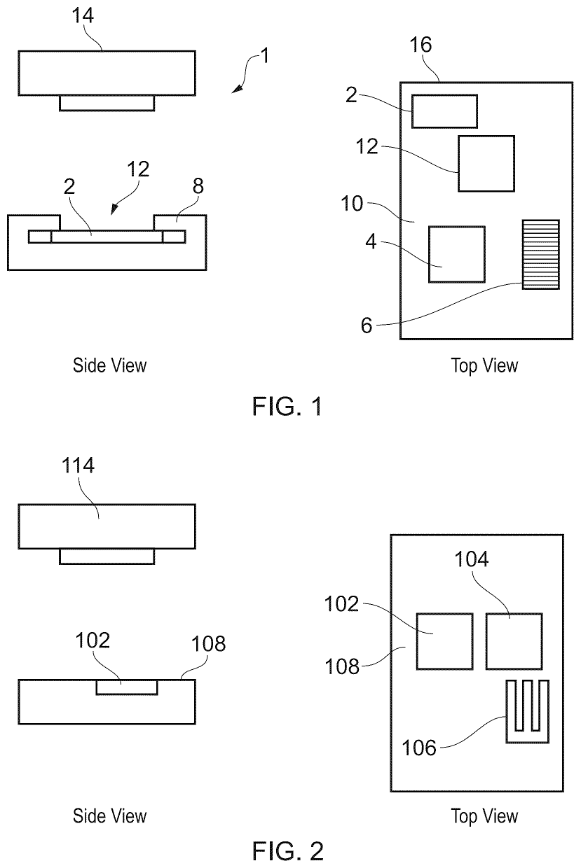

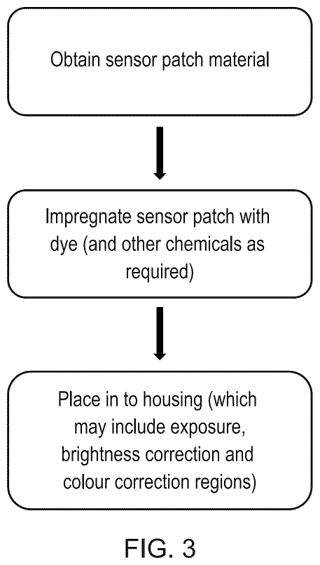

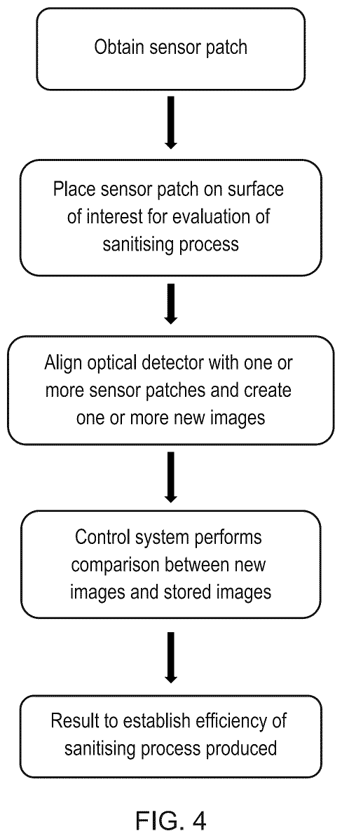

With reference to FIG. 1, a sanitising process detection system 1 comprises a sensor patch 2 for detection of a disinfecting and/or sterilising agent (not shown). The sensor patch 2 is configured to produce a colour change on exposure to disinfecting and/or sterilising agent. The sensor patch 2 is formed from a medium flow rate filter paper material. The material is saturated with 0.5% bromophenol blue (other sensors may use Coomassie Blue®) and a stabilising chemical. The impregnated material is subsequently dried prior to use.

The sensor patch 2 is substantially square in shape. It is however to be understood that the patch 2 may have any suitable shape and or configuration. In the illustrated embodiment, the dimensions of the sensor patch 2 are 2 cm×2 cm×0.18 mm. It is however, to be understood that the patch 2 may have any suitable dimensions depending on the particular requirements for the system 1.

The system 1 further comprises a reference colour region 4. The reference colour region 4 is approximately the colour of the sensor patch 2 and is optionally a colour region that is printed on the housing prior to any exposure to sanitising agents. The reference colour region 4 is placed adjacent (but spaced apart from) the patch 2. It is understood that colours do not have to be identical. The colours are usually similar but a consistent difference in colour is acceptable and provides for a meaningful signal.

The system 1 further comprises a brightness correction region 6 in the form of white and black striped region. The brightness correction region 6 is placed adjacent to and spaced apart from each of the patch 2 and reference colour region 4. In a preferred embodiment a black and white stripped region includes non-symmetrical markers or is in a format which enables orientation of a substrate, on which the stripped region is present, to be detected.

The system 1 further comprises an identifier 16 which may be in the form of alpha numeric lettering or a bar code or a QR code. The identifier may be unique to each substrate or system. The system 1 further comprises a material 8 providing a substrate 10 to support each of the patch 2, reference colour region 4, brightness correction region 6 and unique identifier 16 thereon. The substrate 8 is formed from substrate board having a thickness of 0.1 mm. The substrate in the illustrated embodiment has dimensions of 6 cm×4 cm. It is however to be understood that the substrate 8 may have any suitable dimensions.

The substrate 8 has an aperture 12 located, shaped and dimensioned to enable each of the patch 2, reference colour region 4 and brightness correction region 6 to be visible therethrough. In the embodiment illustrated in FIG. 1, the aperture 12 has dimensioned of 1 cm×1 cm.

The system 1 further comprises an optical detector 14, configured to detect the colour of the sensor patch 2 at one or more predetermined time points. The optical detector 14 is configured to be aligned with a plurality of apertures 12 of the substrate to ensure a line of sight between the detector 14 and each of the patches 2, reference colour region 4 and brightness correction region 6. An array of eight substrates may be imaged simultaneously.

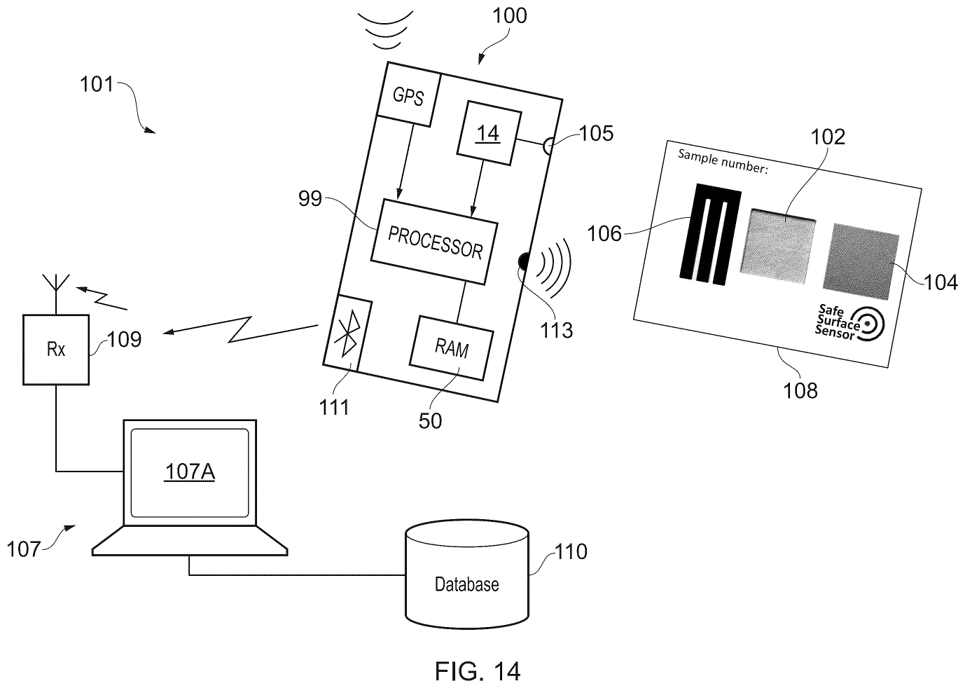

The optical detector 14 of system 1 in the illustrated embodiment in FIG. 14 is provided by the smartphone 100. The processor 99, within the smartphone 100, is configured to execute software for processing image data derived by imager 105 of the substrate. Orientation of image data, location of the sensor patch 102 on the image data and colour analysis of image data is performed by the processor 99 as described in detail below. Data transmission to a user interface 107 is via a hardwire connection or via a wireless connection such as a Bluetooth® protocol wireless device 111 to a receiver 109 connected to the user interface 107. The sensor patch 102 may be illuminated using a light 113 on the smartphone 100.

The imager 105, includes an optical sensor, such as a CCD array or a CMOS array, is configured to capture images of the sensor patch 102 at predetermined time instants, for example continuously. The processor 99 is configured to compare the colour of the sensor patch 2 at a predetermined time instants as determined by the optical detector 14 to the colour of the reference colour region 4. A memory means 50 stores collected images. The system may optionally upload image data to a remote receiver 109 when connected to Wi-Fi or when triggered manually.

With reference to FIG. 14, the system 101 comprises a sensor patch 102 formed of absorbent material (2 cm×1.5 cm) embedded within a substrate 108 formed from paper-based material.

The sensor patch 102 is impregnated with a dye such as Coomassie Blue®. The dye changes colour from light blue to white on exposure to a sanitising antimicrobial agent. The substrate 108 also includes a reference colour region 104 corresponding approximately to the colour of the dye when not exposed to an antimicrobial agent. The substrate 108 also includes a brightness correction region 106.

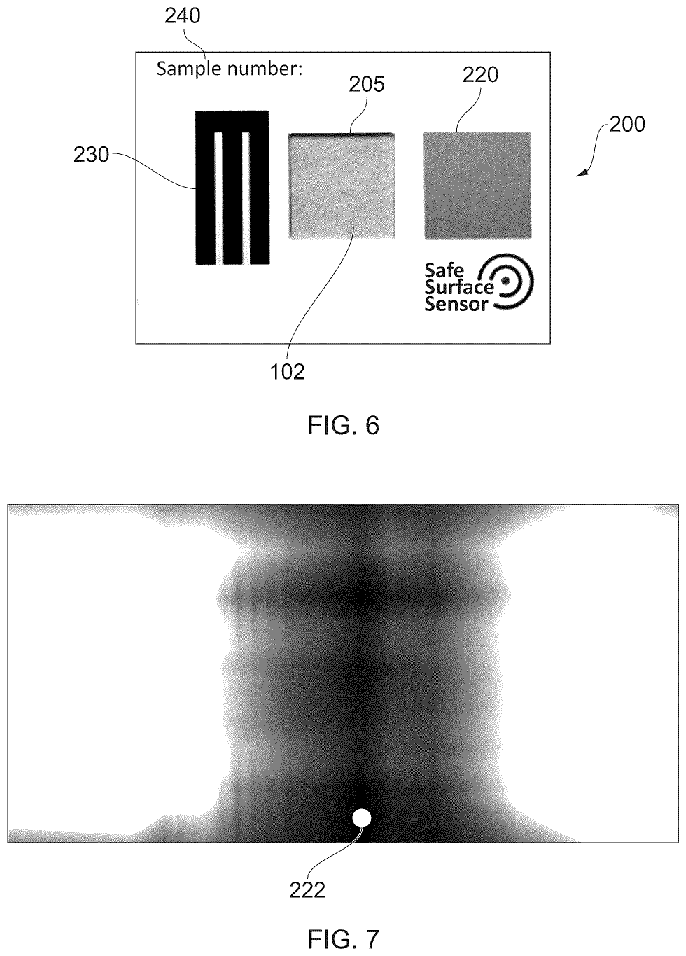

Referring to FIG. 6 there is shown another example of a substrate 200 with a sensor patch or sensor pad 102 thereon. The sensor patch or sensor pad 102 is formed from a piece of absorbent paper (approximately 17 mm×17 mm and around 0.825 mm thick) which is sealed between layers of either synthetic plastics or paper. In some embodiments the layers of either synthetic plastics or paper are formed from a biodegradable material. An upper layer of the synthetic plastics or paper has a square shaped aperture 205 (8 mm×8 mm) which exposes the sensor patch or sensor pad 102. The sensor patch or sensor pad could also be integrated directly into the substrate 200 for example by printing.

Also, on the substrate 200 are a number of printed features. These include a reference colour panel 220 which is used to compensate for different lighting conditions. There is also a registration symbol 230 that assists in orienting the substrate 200 when imaged by software techniques, as described below. There is also a unique identifier 240, which may be an incremented number, bar code or QR code to enable identification of each individual substrate 200, for example when distributed in different locations.

An underside of the substrate 200 has an adhesive strip (not shown) to allow the substrate to be stuck to a surface in order to expose the sensor pad or sensor patch 102.

A wide range of sanitising and disinfecting compounds, which chemically disrupt or interact with a virus or bacteria cell wall, can be detected using the sensor pads or sensor patch 102. These sanitising and disinfecting compounds include quaternary ammonium compounds and any oxidising compound which includes the use of materials that generate free radicals, such chlorine containing compounds and superoxides, such as ozone. The detecting compound is applied to an active part of the sensor pad or sensor patch 102 and is selected to detect a particular sanitising or disinfecting compound.

The sensor patch or sensor pad 102 is dosed with the detecting compound. With addition of the relevant sanitising/disinfecting compound, the detecting compound may change its colour, hue and/or saturation. The detecting compounds may optionally be in the form of dyes or stains. Detecting compounds may be acidic, basic or neutral in nature and may contain particular chemical groups such as: azo, anthraquinone, nitro/nitroso, triarylmethane and indigo groups.

Different detecting compounds are used for different categories of disinfectant. For example, Eosin® can be used to detect for quaternary ammonium compounds and Coomassie Blue® can be used to detect disinfectants containing hypochlorite compounds. The concentration of the detecting compound can be varied to adjust the sensitivity of the sensor pad or sensor pad 102 to a particular disinfectant. The change in the colour or the intensity of colour of the detecting compound can be directly related to the concentration of the sanitising/disinfecting compound that is detected by the sensor pad or sensor pad 102.

The substrate 200 with sensor patches or sensor pads 102 may be produced on a roll (not shown) of flexible material which allows the sensor patches or sensor pads 102 to be distributed using an automatic dispenser. In use substrates, with the sensor patches or sensor pads 102, are distributed at various locations in an area to be sanitised/disinfected. Optionally a record is kept of where the sensor patches or sensor pads 102 are located. The record ideally includes the unique identifier of the sensor patch or sensor pad 102, the time and date the sensor patch or sensor pad 102 is deployed, and optionally, an automated position record which may be obtained for example using a global positioning system (GPS). This may assist in retrieval of the substrate as well as providing a record of the location of where each substrate is deployed.

The sanitisation/disinfection process then typically commences and following exposure to a disinfectant the sensor patches or sensor pads 102 are collected. They are typically placed in an array for the imaging means or imager 105 to image all sensor pads 102 at once.

A reusable plastics template (not shown) may be used for the collection of the sensor pads. Markers may be placed on the reusable plastics template in order to assist in positioning of each of the sensor pads 102 in an image frame.

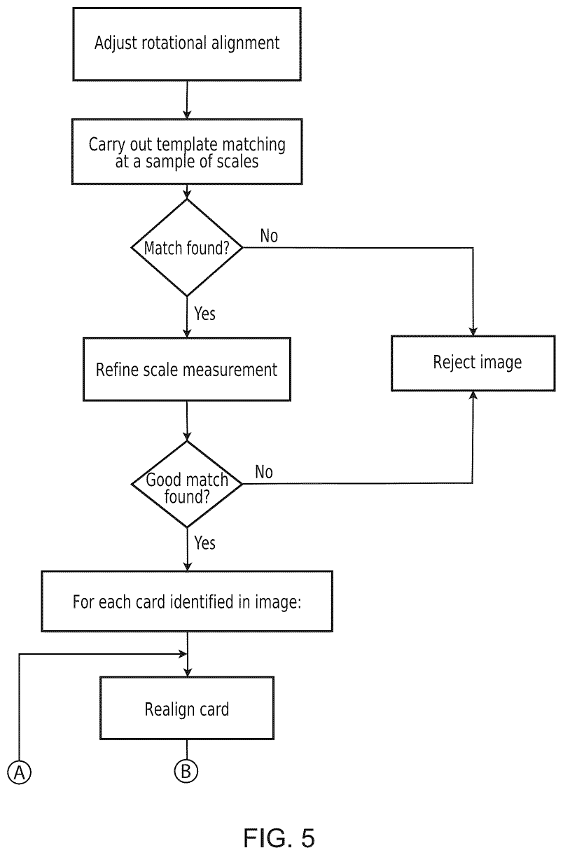

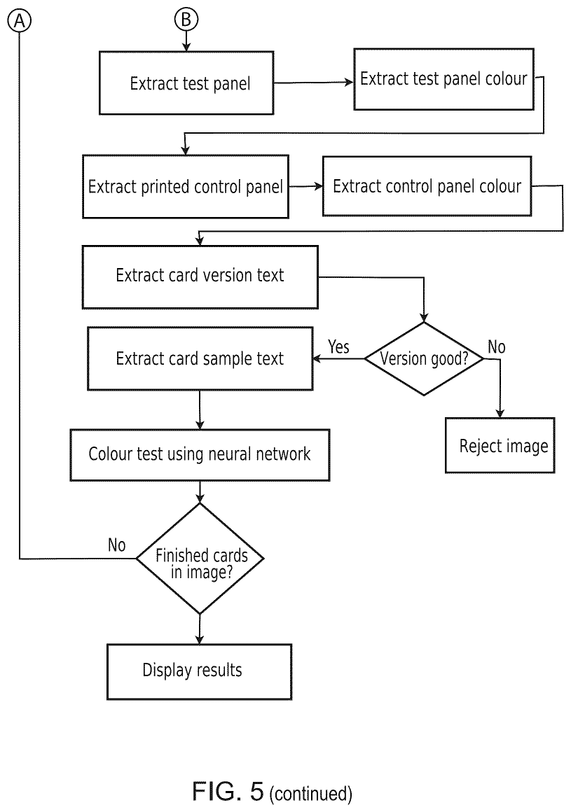

FIG. 5 shows an example of a flow diagram of a preferred embodiment of the method showing steps of image alignment, colour testing using a neural network to assess the sanitisation process. In the system 101, which has an imager 105 which is housed in a smartphone, suitably modified with an application specific software (APP) to process images obtained with a smartphone camera. The smartphone also provides the user with an ability to connect to an Internet site and send and receive data to a remote location.

The system 101 comprises an optical detector 114, which includes an imager 105 and the processor 99, which is incorporated in a smartphone configured to detect the colour of the sensor patch 102 at one or more predetermined time instants. The optical detector 114 is configured to be aligned with the aperture 112 of the substrate to ensure a line of sight between the detector 114 and each of the patch 102, reference colour region 104 and brightness correction region 106.

The system 101 in the illustrated embodiment is shown as a flow diagram and is provided by a processor within the smartphone. The processor is configured to execute software for image processing, colour analysis, data transmission and a user interface. The processor is configured to determine a colour change of the sensor patch 102 at a predetermined instant which may be determined by a user or by an alert issued from a control means. The alert prompts a user to retrieve one or more of the substrates, optionally in a prescribed order, and to place them on the reusable plastics template.

Referring to FIGS. 1 and 14, an imager or imaging means 105 and processor 99 operate in accordance with software (in an APP) in order to supervise imaging processes such as edge detection, orientation, size scaling, image alignment and colour comparison of region 104 with a reference colour. In addition a neural network continually updates learning software in order to improve image analysis.

An advantage of the embodiment, in which a smartphone is used, is that images of substrates that may be hidden or positioned in discreet locations, may be obtained quickly and easily. Because the software enables the processor to detect edges, orient image data frames and better perform colour comparisons in varying lighting conditions, a user is able to use a smartphone (or a similar customised hand held device) to obtain images in inaccessible locations or in places that inhibit images of substrates to be obtained easily. Even when images are obtained, from a non-orthogonal (skewed) distance from a substrate and/or when they inverted (‘upside-down’) with respect to the imager 105, the software effectively standardises image data in order to provide a consistent and reliable output of the extent to which sanitation has taken place. At the same time an authenticated record of the location of an imaged substrate is simultaneously obtained due to the automatic time and date stamping of an image data frame. Optionally the GPS location of a substrate may also be captured and stored. This hand held embodiment of the system therefore enables many substrates to be imaged quickly and consistently.

The reference colour may be a datum determined from a database of images of sensor patches including one or more sensor patches prior to and/or post exposure to the disinfecting and/or sanitising agent, or a datum which is provided as a reference colour region on the substrate 104.

The optical sensor comprises an array of complementary metal-oxide-semiconductors (CMOS) sensors which gather an image of one or more of the substrates and their respective sensor patches. The APP may have a secure login so that secure registration of users is ensured. The APP performs a number of functions and may operate in conjunction with a database 110 of stored image data files to determine the presence of a colour change.

The following are some functions performed by the APP.

The APP identifies a sensor pad in the aperture from the images of substrate with sensor pads.

The APP obtains and calculates changes in colour, hue and/or saturation of the detecting compound.

The APP supervises access to a neural network to assess changes in colour, hue and/or saturation of the detecting compound.

The APP displays results pictorially, for example on a display or screen 107A in a simple format, such as ‘PASS’ or ‘FAIL’.

The APP produces and stores lists of results and outputs.

The APP oversees communication with an Internet or supervisor at user interface 107.

The APP allows menus to be established and defined for different applications and users.

Template matching is described below. Once an image has been captured, the first step is to locate the substrates in the image. For this, a technique called template matching is used, which tries to match parts of the image with an example image of a substrate, called a template.

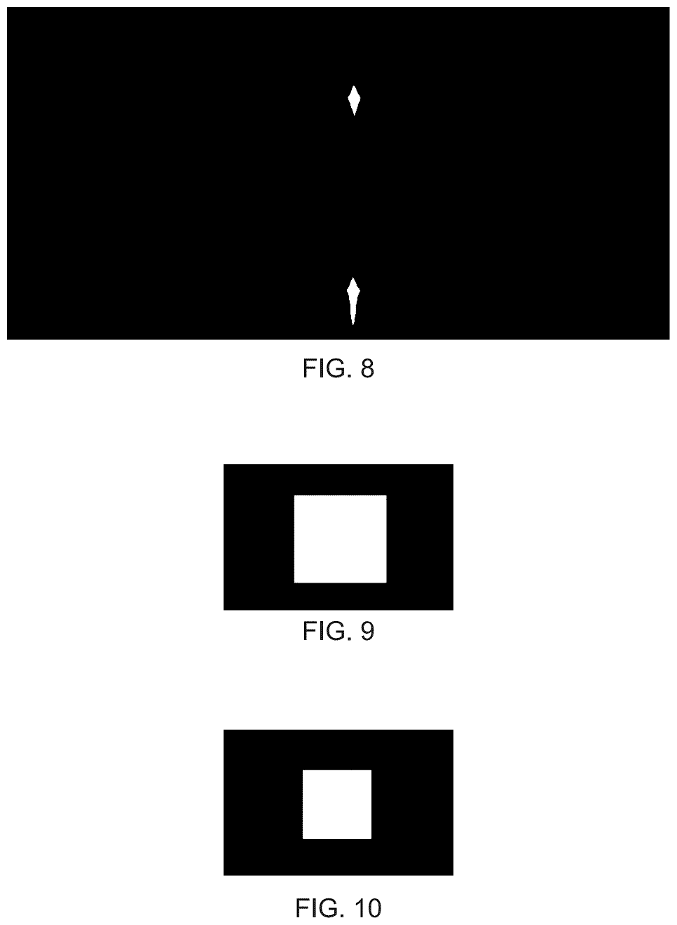

Referring to FIGS. 6 to 10 the template 200 is positioned in a particular location and is imaged. Image data is conceptually placed, by way of software manipulating the image data derived from a CMOS device in imager 105. Orientation is achieved with reference to a top left hand corner of an image as described below. A region covered by the template is compared by subtracting each pixel value in the template from a corresponding pixel value in the image. If that portion of the image is identical then all the resulting pixels would be zero after this subtraction. Hence by looking for zeroes the substrate in the top left hand corner of the image is identified. A simplified YES or NO result is obtained by adding together all of the pixel subtraction values. This can be graphically represented by putting an overall sum, for example in a top left hand corner of a frame as a pixel value. However in practice the process is more complicated because, even if there was a representation of the substrate in the top left hand corner of the image, it would not be identical to the template image of the substrate as some pixels in the image copy of the substrate may be brighter and others darker because of ambient lighting and variations in panel colour and shadow effects.

So, rather than simply summing differences of pixel values, the squares of differences between pixel values and a datum are summed as that value is always positive. This also has an additional benefit that large differences are squared, thereby penalizing values that are markedly different to a greater extent than smaller differences. The number that is obtained is therefore a measure of how much that portion of the image resembles the substrate template.

Hence, this acts as a detector for the top left hand corner of the image being also the top left hand corner of a substrate. If this operation is repeated for all pixels in the image. Following this stage a value of each pixel in the image is derived that represents how close it matches the top left hand corner of a substrate. The smaller the number, the closer to being a substrate image it is. A threshold value is then set below which a substrate has been detected in the image.

FIG. 7 shows an example of a template matching pattern for the template slid over the image. Darker areas represent better matches. The dark dot 222 indicates the best match position in the entire image. The scan does not cover the pixels at bottom and right side of the image where the template would run over the edge of the image. Darker colours signify smaller pixel values, and thus indicate better match with the template. Pixels mostly have non-zero values. Therefore a threshold has been applied to clearly differentiate them.

Template matching is carried out using monochrome images. A grayscale image is far faster to compare than a colour one, because each pixel is represented by an 8 bit value instead of the three 8 bit values for red, green and blue. It is important that substrates in the image are rotatable. This is because if a user does not image a patch in a perfectly upright orientation errors are introduced. Therefore image processing software effectively rotates image data so that correctly orientated data is extracted from a from an image frame for processing. Therefore prior to an image being processed, to extract colour match data, orientation of image frame data is checked.

As printed graphics on a substrate are rectilinear, with vertical and horizontal lines, in an image of several substrates (or a single substrate) vertical and horizontal lines should dominate. This assumption fails if any background contains diagonal lines, for example if substrates are mounted in a skewed configuration with respect to vertical and horizontal.

In order to mitigate this problem a Hough Transform algorithm is used to identify straight lines in the image data. As shown in the flow diagram in FIG. 5, the processor checks that detected straight lines are vertical, or nearly vertical, and derives an average angle of deviation of these lines is. This angle of deviation is treated as an estimate of how much the image is rotated out of alignment. If the image is out of alignment, then it is rotated digitally so that any off vertical lines are vertical.

As images are not always obtained from the same camera to substrate spacing, the size of the substrates in the image are not always the same—they may be smaller than the template, or larger than it.

The template is deemed to provide a reasonable match with a substrate in the image if it is approximately the same size as the substrate in the image. If the two images differ by a predefined amount, then the match is considered as poor and may be rejected. Hence the program carries out a short series of template matches using the template scaled to different sizes. The template is scaled rather than the image because it is computationally cheaper to scale the template than the image. A modified version that is normalized of the square-of-differences algorithm is used to compare values produced by different sized templates.

The program generates an internal table of the single best template match pixel result for each scale. It then refines scale by looking at the closest three matches and using the Nelder-Mead algorithm to carry out further matches iteratively in an optimum fashion at different template scalings in order to obtain the precise scale of template that gives the best match with the image. Therefore a Nelder-Mead technique is used because template matching is computationally expensive, and this helps to minimise calculations and to reduce processing time.

Once the scale of the substrates in the image has been determined it is necessary to accurately identify the top left hand corner of the substrates. For the final step a further template match is done at the now correctly determined scale, but using a more sophisticated (and computationally expensive) algorithm to cut down the number of false positive matches. This algorithm multiplies each point with the corresponding template value, normalizes the results and then works out the Pearson correlation coefficient between the template and the portion of the image being matched.

Since the template does not have to be exactly aligned with the substrate to produce a reasonable match, the final result shows regions where the substrates start in the image rather than specific points (see FIG. 5). These must be converted to single points. A blob detector provides the central point of regions in an image that differ from the surrounding regions, and this is used to identify blobs that contain the top left hand corners of the substrates. Once the blobs have been defined, the program picks the pixel within each one with the best match value. These are then used to indicate the corner of the substrates.

Scaling the template is performed as required so that size of the image in which the substrate is located is the same size as the original template image. This is an important step, since image data can then be processed without having to worry about their sizes since all images processed in this way have the substrates the same size. Colour processing may also be applied at this stage.

Sometime features may need to be extracted. The features that may be needed from each substrate are found in image data. In some systems this is done by creating a pool, for example consisting of ten data sets of separate execution threads to examine each substrate so that several substrates in the image can be examined in parallel if the microprocessor in the smartphone supports this.

If there are less than ten data sets in the image, then the first ten substrate data sets are processed in parallel. If there are more than ten data sets then ten at a time, with the next substrate being processed as a worker thread becomes idle after processing the previous substrate.

There are four different features that are required from the substrate:
Sensor pad colour/hue/saturation
Printed control panel colour
Substrate version number string
Substrate sample number

The overall strategy in each case is to define a region within the substrate image that is to be searched for the feature. This is to prevent other substrate features from being mistaken for the feature itself. The search area is always larger than the feature since the location of the feature in the image of the substrate can change slightly if the substrate is not photographed directly on, i.e. with the axis of the camera lens absolutely perpendicular to the substrate.

For the aforementioned reasons, it is strongly desirable that the features are separated by white space on the substrate. The search area is implemented is done by defining a mask, and cutting out the portion of the image that corresponds to the mask. This portion is then searched. During this process, partial substrate images, i.e. ones that run off the right or bottom of the photograph, are discarded.

Locating the sensor patch within a search area can be challenging as substrates may not always be imaged in a precisely rectilinear format. One problem with multiple substrates in an image is that some images may have been rotated. To correct this image data from substrates have to be aligned horizontally. This alignment process is performed on image data before template matching is carried out. As before detection is performed by looking for vertical lines in the template part of the image.

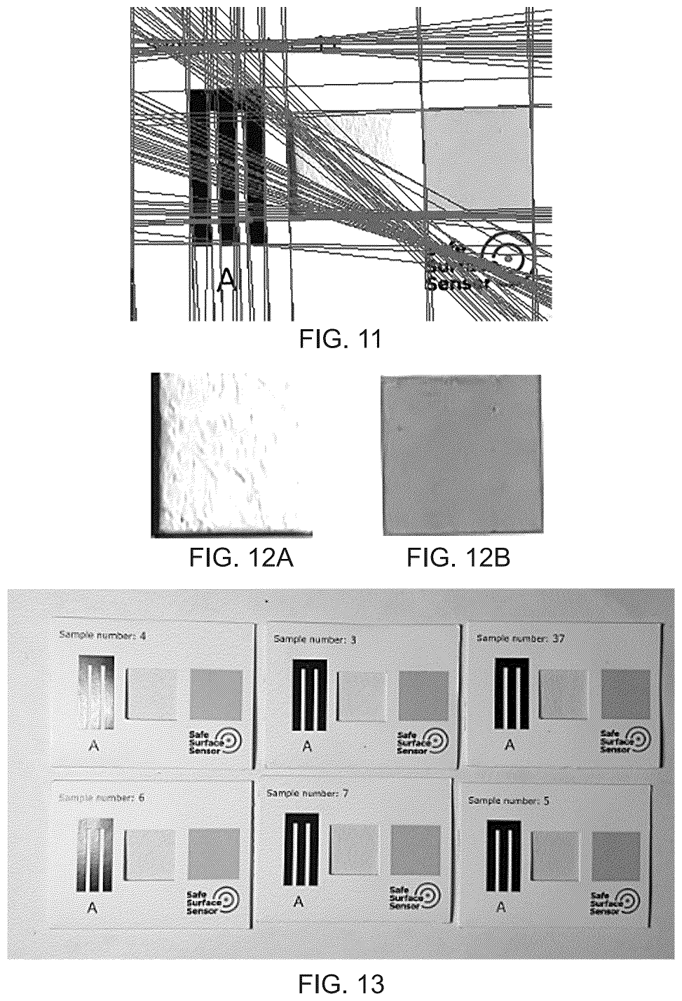

FIG. 11 shows lines obtained using alignment software to orient image date derived from a substrate. Defining characteristics are changes in colour around an outside portion of the substrate, transitioning from a white of the substrate to the colour of the panel. Alternatively from a black line around a panel if one is present. Imaging software therefore seeks contours in a search area of the panel. One of these should be a boundary or panel edge. There may be others, for example representing contours within the colour panel.

A difficulty encountered is to extract a correct contour that defines an actual boundary of the panel. Some of the contours may be due to colour changes in the white substrate outside of the panel caused by illumination. The problem is overcome in a preferred embodiment by operating a processor to apply different algorithms for different sanitising agents.

Imaging software code takes each contour line, and fits a minimum area rectangle around it. It orders the contours according to the minimum difference between the contour length and this bounding rectangle, which corresponds to the most rectangular contour. The reasoning here is the bounding contour of the panel is rectangular to match the actual shape of the panel.

It then goes through the list of bounding rectangles and tests each one in turn until it finds the contour that is within limits for the size matching the expected size and squareness of the panel being searched for. The panel is square, so the minimum rectangle around the corresponding contour should also be approximately square.

Imagine that part of the black “cricket wicket” symbol 230 or 106 to the left of the test panel is included in the image. In this case, there is a contour around the sensor patch but this is discarded because it is neither square nor the correct perimeter length.

Referring to FIGS. 12A and 12B there are shown two panels located in substrate 12. The two text fields, the substrate version number and the sample number, are extracted. The search area is passed to an Android text recognition module that decodes text. This internally works by splitting the region into its discrete blobs and using a neural network to recognize each blob as a letter. The version string is checked to see if it matches the expected version string for the substrate type.

Once the features have been extracted the final stage is to examine the colour of the sensor patch area. This is a difficult problem because the illumination is not under the control of the APP. Required inputs are the colour of the test panel and as much information about the type of substrate illumination as possible. Because it is not a well understood problem where statistics can be used, the best approach is one of machine learning. The sensor patch colour is taken to be the dominant colour of the test panel. It uses a k-mean clustering algorithm to create a single cluster whose mean is the mean colour of the panel.

It creates four different values:
The mean red/green/blue (RGB) values of the test panel
The mean hue saturation value (HSV) values of the test panel
The difference between the mean RGB values of the test panel and the mean RGB values of the control panel, this difference changes according to illumination conditions.
The mean RGB values of the whole substrate.

The last of these, the mean RGB value of the whole substrate is just used to decide against threshold values whether the illumination is satisfactory, if not the substrate test is not carried out. At present the threshold is set so that the test may be performed under any illumination conditions. This is however intended to prevent the need for the test to be performed in, for example, almost pure red monochromatic light. Optionally software manipulates image data sets to provide a standardised colour irrespective of ambient lighting conditions.

A neural network is used to classify the substrate colour according to first three of the values above. One neural network is provided for each type of sanitizing/disinfecting compound. The neural networks are all based on a Keras module and trained in a process described below. Keras is a library of Python® modules to facilitate creating and training machine learning systems.

Below is a description of how confusion matrices (such as test results) for a neural network. These are:

### Disinfectant A

(True labels on Y axis, predicted on X axis)

| | exposed | unexposed |
|---|---|---|
| exposed | 0.99 | 0.01 |
| unexposed | 0.01 | 0.99 |

### Disinfectant B

(True labels on Y axis, predicted on X axis)

| | exposed | unexposed |
|---|---|---|
| exposed | 0.99 | 0.01 |
| unexposed | 0.01 | 0.99 |

### Disinfectant C

(True labels on Y axis, predicted on X axis)

| | exposed | unexposed |
|---|---|---|
| exposed | 1.0 | 0.0 |
| unexposed | 0.0 | 1.0 |

Every time a test or release version of a test code is built, it is tested using a regression test set. For example, this could be 50 image files, each containing around 240 substrates. This could include some partial substrate images and some rotated images, so not all are expected to ever be processed successfully. The main purpose of the regression test is to tell if a code change has “broken” a rule of the neural network.

Neural networks are trained in a three stage process. The first stage is to generate a training set of image data. A separate APP controls the smartphone camera and a Philips Hue® lightbulb. The smartphone is positioned over an array of exposed substrates illuminated by the Philips Hue® bulb and the APP run in near-complete darkness.

Imaging software cycles through different colours using the Philips Hue® bulb, taking a photograph at each colour. This process can take several hours and produces approximately 7000 images, each one containing multiple substrates. The number of images can be adjusted by changing the step size used in the Hue light control code. The process is then repeated with an array of unexposed substrates.

For the second stage of training, characterising each member of the training set, one of the substrate sets is loaded onto a smartphone running the APP, into a digital camera imaging (DCIM) directory. The DCIM directory is used for regression testing, and normally contains a standardized sample of substrate image data.

A DUMP_COLOUR_DATA debugging flag is set and the APP is run. This initiates processing of all image data. Because the DUMP_COLOUR_DATA flag is set the APP appends colour data for each set of image data it identifies into a CSV file called colours.txt. An example of the file is shown below:

Question marks are used where the substrate sample number cannot be read, and so are dismissed as not usable as data sets for training.

Two files are produced by running the APP on the images for the substrates treated with disinfecting compound and the untreated substrates. The CSV files are then processed by the Keras Python script that defines the neural network and carries out the training. The output from the script is a model file for each disinfecting compound that is incorporated into the APP (and used as described above).

A neural network is ideally configured to be trained to distinguish between treated and non-treated sensor patches.

The system is configured to capture images of the patch at predetermined time instants, for example continuously; store the collected images; extract reference marks from the images; extract unique identifiers (such as a QR code, a barcode, alpha numeric or symbols) from the patch or substrate; identify reference colours; extract colour or brightness data from reference colours; extract relevant descriptive statistic from patches for determining if the sanitising process has reached a minimum acceptable level; apply thresholds to the patch images or through the use of a neural network to determine whether the image is one of a treated or untreated patch; display the result; store the result. The system may upload the data and images when connected to Wi-Fi or when triggered manually.

In use, the optical detector 14, 114 monitors the colour of the sensor patch 2, 102 at predetermined time intervals, or continuously, depending on the requirements for the system 101.

The optical detector 14, 114 takes images of the colour of the sensor patch 2, 102 at the required time intervals and provides these to the system. The system compares the colour images provided by the optical detector 14, 114 to the colour of the reference sample 4, 104. The system is configured to detect a change in colour of the sensor patch 2, 102 compared with a reference sample 4, 104 and provides an indication to the user when the colour of the sensor patch 2, 102 has changed to a predetermined colour indicative of exposure to a disinfecting/sterilising agent.

The method analyses the sensor patch 2 and assesses whether or not a sensor patch 2 has been exposed to sanitising agents. The method includes the steps of: obtaining a signal required by a neural network programme for example, the outputs of the Red/Green/Blue channels, the hue, saturation and value channels of the imager and/or, the differences between the values from the sensor pad and printed reference colour.

Training of the neural network is carried out under a range of lighting conditions encompassing different light intensities and colour temperatures.

The system of the present invention can therefore provide an indication as to when an area, for example a room or other space or surface, has been effectively sanitised. That is whether a space or surface has been exposed to at least a predetermined minimum amount of a disinfectant/sterilising agent.

The patches may be changed routinely as a result of colour changes. The system provides an accurate and effective means for ensuring that areas are being effectively sanitised.

It will be appreciated that variation may be made, without departing from the scope of protection, as defined by the claims.

1. A sanitising process detection system comprising:
a substrate with a sensor patch which is impregnated with a chemical which produces a colour change on exposure to a disinfecting and/or sterilising agent;
an optical detector includes a processor and an optical sensor which is operative to detect the colour change; and
the processor is operative to compare the colour change with a datum to indicate efficacy of the sanitising process.

2. A sanitising process detection system comprising:
a substrate with a material coated thereon, the material has at least one chemical which produces a colour change on exposure to a disinfecting and/or sterilising agent;
an optical detector includes a processor and an optical sensor which is operative to detect the colour change; and
the processor is operative to compare the colour change with a datum to indicate efficacy of the sanitising process.

3. A sanitising process detection system according to claim 1, wherein the datum is a reference colour region on the substrate.

4. A sanitising process detection system according to claim 1, wherein the optical detector is operative to determine an orientation of the substrate.

5. A sanitising process detection system according to claim 1, wherein the optical detector is operative to determine a location of the sensor patch.

6. (canceled)

7. A sanitising process detection system according to claim 1, wherein the substrate has an orientation marker.

8-10. (canceled)

11. A sanitising process detection system according to claim 1, wherein the at least one chemical is a dye which provides a colour change on exposure to quaternary ammonium compounds.

12. A sanitising process detection system according to claim 1, wherein the processor includes an edge detector.

13. A sanitising process detection system according to claim 1, wherein the processor includes an exposure correction means.

14-15. (canceled)

16. A sanitising process detection system according to claim 1, wherein the optical detector is included in a smartphone.

17. (canceled)

18. A sanitising process detection system according to claim 16, wherein a position signal, such as a GPS signal data, indicative of the location of the substrate is, associated with a score efficacy of the sanitising process.

19. (canceled)

20. A sanitising process detection system according to claim 1, in which the system further comprises a scaling step in which images are scaled to a standard size.

21. A sanitising process detection system according to claim 1, in which the system further comprises an indication mechanism operable to provide an indication as to the effectiveness of the sanitising process dependent on the colour of the sensor patch.

22. A method of assessing the efficacy of a sanitising process comprising the steps of: positioning at least one substrate which has a sensor patch which produces a colour change on exposure to a disinfecting and/or sterilising agent, the substrate has an identifier; associating the defined location of the at least one substrate with its identifier; subjecting the at least one substrate to a sanitising process for a defined time period; retrieving the at least one substrate and using an optical detector to detect a colour change of the sensor patch to derive an indication of the efficacy of the sanitising process at the location at which the substrate was positioned.

23. A method according to claim 22, includes the step of: recording the location of each substrate and recording a score indicative of the efficacy of the sanitising process at the location at which the substrate was positioned.

24-27. (canceled)

28. A method according to claim 22 includes the step of using a neural network to obtain an improved result from at least one data set.

29. A method according to claim 22, includes the step of storing the score indicative of the efficacy of the sanitising process and/or the time and date stamp and/or location coordinates in a memory means, such as a remote database.

30. (canceled)

31. A substrate for use with a sanitising process detection system, the substrate a material coated thereon with at least one chemical which produces a colour change on exposure to a disinfecting and/or sterilising agent, characterised in that the substrate has an identifier.

32. (canceled)

33. A substrate according to claim 31 wherein the substrate has an orientation marker which enables an optical detector to orient and locate the sensor patch.

34-35. (canceled)

36. A substrate according to claim 31 wherein the substrate further comprises at least one aperture configured to provide visual access to the sensor patch.
